(19) United States
(12) Patent Application Publication
(10) Pub. No.: US 2022/0074908 A1
(43) Pub. Date: Mar. 10, 2022

(54) GROWTH MONITORING SYSTEM

(71) Applicant: Radient Technologies Innovations Inc., Edmonton (CA)

(72) Inventors: Michael Cabigon, Edmonton (CA); Jim Seethram, Edmonton (CA); Steven Splinter, North Vancouver (CA); Denis Taschuk, Edmonton (CA)

(21) Appl. No.: 17/238,050

(22) Filed: Apr. 22, 2021

Related U.S. Application Data
(63) Continuation of application No. PCT/IB2019/058797, filed on Oct. 15, 2019.
(60) Provisional application No. 62/749,072, filed on Oct. 22, 2018.

(51) Int. Cl. G01N 33/00

(52) U.S. Cl. CPC G01N 33/0098; G06Q 50/02

(57) ABSTRACT

The present disclosure is directed to collecting and evaluating plant growth data and to making estimates from the plant growth data. Estimates that may be made from the plant growth data identify a total volume or mass of plant matter that is projected to be yielded from cannabis plants growing at one or more farms. Estimates made by methods and apparatus consistent with the present disclosure may identify a mass or volume of cannabis concentrates that could be created by an extraction process that extracts cannabinoids from an volume of cannabis plant matter. Methods and apparatus consistent with the present disclosure may also allow a computer of a manufacturer to receive data from either a grower computer or from a computer of an extractor such that a manufacturer can arrange to purchase cannabinoid containing extracts that may be incorporated into products that may be consumed or used by a person.

### CROSS-REFERENCE TO RELATED APPLICATIONS

The present application is a continuation of PCT/IB2019/058797 filed on Oct. 15, 2019 which claims priority benefit of U.S. provisional patent application No. 62/749,072, filed on Oct. 22, 2018, the disclosures of which are incorporated herein by reference.

### BACKGROUND OF THE INVENTION

### 1. Field of Invention

The present disclosure is generally directed to collecting data that allows growers to optimize the growth of cannabis plant matter for an extraction process. More specifically, the present disclosure is directed to cross-referencing data collected by a farmer with extraction data that could allow manufacturers of cannabinoid containing products to purchase cannabis concentrates that are consistent with edible product requirements.

### 2. Description of the Related Art

The term cannabis or “cannabis biomass” encompasses the Cannabis sativa plant and also variants thereof, including subspecies sativa, indica and ruderalis, cannabis cultivars, and cannabis chemovars (varieties characterised by chemical composition), which naturally contain different amounts (ratios or masses) of the individual cannabinoids, and also plants which are the result of genetic crosses. The term “cannabis biomass” is to be interpreted accordingly as encompassing plant material derived from one or more cannabis plants of certain types of cannabis plants.

Cannabis plants or plant biomass contain a unique class of terpeno-phenolic compounds known as cannabinoids or phytocannabinoids. The principle cannabinoids present in most cannabis variates are the Delta-9-tetrahydrocannabinolic acid (THCA) and cannabidiolic acid (CBDA). The THCA does not have its own psychoactive properties as is, but may be decarboxylated to Delta-9-tetrahydrocannabinol (THC), which is a potent psychoactive cannabinoid. The neutral or non-acidic form of CBDA is cannabidiol (CBD), which is a major cannabinoid substituent in hemp cannabis. CBD is non-psychoactive and is widely known to have therapeutic potential for a variety of medical conditions.

The proportion of cannabinoids in the plant may vary from species to species, as well as vary within the same species at different times and seasons. Furthermore, the proportion of cannabinoids in a plant may further depend upon soil, climate, harvesting time, and harvesting methods. Thus, based on the proportion of the cannabinoids present in a plant variety, the psychoactive and medicinal effects obtained from different plant varieties may vary. Such variance is further exacerbated by the presence of certain terpenoid or phenolic compounds which may be present in the plant, which may also have pharmacological activity.

Historical delivery methods have involved smoking (e.g., combusting) the dried cannabis plant material. Smoking results, however, in adverse effects on the respiratory system via the production of potentially toxic substances. In addition, smoking is an inefficient mechanism that delivers a variable mixture of active and inactive substances, many of which may be undesirable. Alternative delivery methods such as ingesting typically require extracts of the cannabis biomass (also known as cannabis concentrates or cannabis oils). Often, cannabis extracts are formulated using any convenient pharmacologically or food-grade acceptable diluents, carriers or excipients to produce a composition. Collectively, product made from such extracts may be known as cannabis derivative products or cannabis products that may be in the form of a consumable item or may be in the form of a balm or rub that may be applied to the skin as a topical agent. As such, cannabis edibles may be in the form of a food product such as a chocolate, a cookie, or as a beverage or as an oil intended to be ingested, such as in a capsule. Other cannabis products may be in a form that is readily vaporized in a vaporizer or that is in a form that is readily absorbed through the skin. Often cannabis is grown by farmers that may be referred to as “growers.” Cannabis extracts and derivative products produced by entities that extract cannabinoids from plant matter may be referred to as “extractors.” Entities that combine concentrates into a product that is consumed by persons may be referred to as “cannabis product processors or manufacturers.” In some instances, two or all three of the growers, extractors and product processors may be the same entity.

Cannabis extracts may be obtained from cannabis biomass by any number of methods, including but not limited to supercritical fluid extraction or solvent extraction of microwave-assisted extraction. In most cases, the yield of cannabis extract obtained, and thus the yield of final cannabis derivative product obtained from the raw cannabis biomass will depend upon the composition of the cannabis biomass used for the extraction, including for example the potency or concentration of cannabinoids present in the cannabis biomass. In some cases, the yield of cannabis extract and the quality of cannabis extract may be different depending on the extraction conditions used to obtain the extract. For example a solvent type, a ratio of solvent to biomass, or a temperature and time of extraction may each cause the quality of an extract to vary. The quality of the cannabis extract may be dictated by the potency or concentration of cannabinoids in an extract, the cannabinoid profile in the extract (i.e the relative concentrations of various cannabinoids present), or a terpene profile in the extract (i.e. relative concentrations of various terpenes present). The quality of the cannabis extract may also be dictated by the physical properties of the extract, including but not limited to color or viscosity. In some cases, cannabis product processors may desire cannabis extracts with particular chemical and physical properties and profiles that may be preferred for different product types and forms.

There is a need to assist the cannabis extractor to use data of past growth and extractions of various plant types and cultivars to create the best process for a current extraction. There is also a need to use growth data for supply chain management of cannabis extract and cannabis product manufacturing.

### SUMMARY OF THE CLAIMED INVENTION

The presently claimed invention relates to a method, a non-transitory computer readable storage medium, or an apparatus executing functions consistent with the present disclosure. A method consistent with the present disclosure may include receiving cannabis plant growth data, identifying a type of cannabis plant from the cannabis plant growth data, estimating an amount of the cannabis plant matter, and estimating a mass of cannabinoids and other compounds that can be extracted from the estimated amount of cannabis plant matter.

When the method of the presently claimed invention is implemented as a non-transitory computer-readable storage medium a processor executing instructions out of a memory may receive cannabis plant growth data, identify a type of cannabis plant from the cannabis plant growth data, estimate an amount of the cannabis plant matter, and estimate a mass of cannabinoids and other compounds that can be extracted from the estimated amount of cannabis plant matter.

An apparatus consistent with the present disclosure may include a memory and a processor that executes instructions out of a memory to receive cannabis plant growth data, identify a type of cannabis plant from the cannabis plant growth data, estimate an amount of the cannabis plant matter, and estimate a mass of cannabinoids that can be extracted from the estimated amount of cannabis plant matter.

### BRIEF DESCRIPTIONS OF THE DRAWINGS

FIG. 1 illustrates an exemplary network environment in which an exemplary system of using growth-based metrics for extraction optimization may be implemented.

FIG. 2 is a flowchart illustrates an exemplary method for generating growth-based metrics regarding a cannabis grow site.

FIG. 3 is a flowchart illustrates an exemplary method for identifying extraction parameters for specified types of cannabis plants.

FIG. 4 is a flowchart illustrates an exemplary method for identifying extraction parameters for specified cannabis plant biomass.

FIG. 5 is a flowchart illustrates an exemplary method for calculating yield estimates based on grow data.

FIG. 6 illustrates a computing system that may be used to implement an embodiment of the present invention.

### DETAILED DESCRIPTION

This present disclosure is directed to promoting efficiency of the cannabis extraction process based on ample data provided by the service platform that monitors growth of cannabis across cannabis biomass types and cultivars and monitoring of growth conditions. This invention also facilitates better management of manufacturing resources by providing estimates in the yields and quality of cannabis concentrates and information collected in the extraction process.

The term cannabis or “cannabis biomass,” “cannabis plant matter biomass”, cannabis plant matter,” or simply “biomass” includes the cannabis sativa plant and also variants thereof, including subspecies sativa, indica and ruderalis, cannabis cultivars, and cannabis chemovars (varieties characterised by chemical composition), which naturally contain different amounts of individual cannabinoids. These terms may also be assigned to cannabis plants that are the result of genetic crosses of one or more subspecies. The term cannabis is to be interpreted accordingly as encompassing plant material derived from one or more cannabis plants. The term “cannabis extract” or “extract” encompasses any extract of the cannabis biomass (also known as cannabis concentrate, cannabis oil, cannabis distillate, cannabinoid crystals, or cannabinoid isolates). A cannabis concentrate may be a product that has been extracted from cannabis plant biomass that may include a higher concentration of cannabinoids per unit mass than a concentration that exists in the cannabis plant biomass itself. Cannabis oil or distillate may be a concentrate that includes waxy substances or that may also include plant terpenes that were extracted from the cannabis plant biomass. As such, cannabis oils or cannabis distillates are not pure or substantially pure. Commonly such oils or distillates may contain somewhere between 50% and 80% cannabinoids per unit mass of the oil or distillate. Cannabinoid crystals, however, can contain nearly pure, greater than 95% cannabinoids per unit mass of the crystal. Cannabinoid isolates may only one type of cannabinoid that is nearly pure (greater than 95%). As such an isolate of cannabidiol (CBD) could contain 95% to 100% CBD.

The present disclosure is directed to collecting and evaluating plant growth data and to making estimates from the plant growth data. Estimates that may be made from the plant growth data identify a total volume or mass of plant matter that is projected to be yielded from cannabis plants growing at one or more grow sites. Estimates made by methods and apparatus consistent with the present disclosure may identify a mass or volume of cannabis concentrates that could be created by an extraction process that extracts cannabinoids from an volume of cannabis plant matter. Methods and apparatus consistent with the present disclosure may also allow a computer of a manufacturer to receive data from either a grower computer or from a computer of an extractor such that a manufacturer can arrange to purchase cannabinoid containing extracts that may be incorporated into products that may be consumed or used by a person.

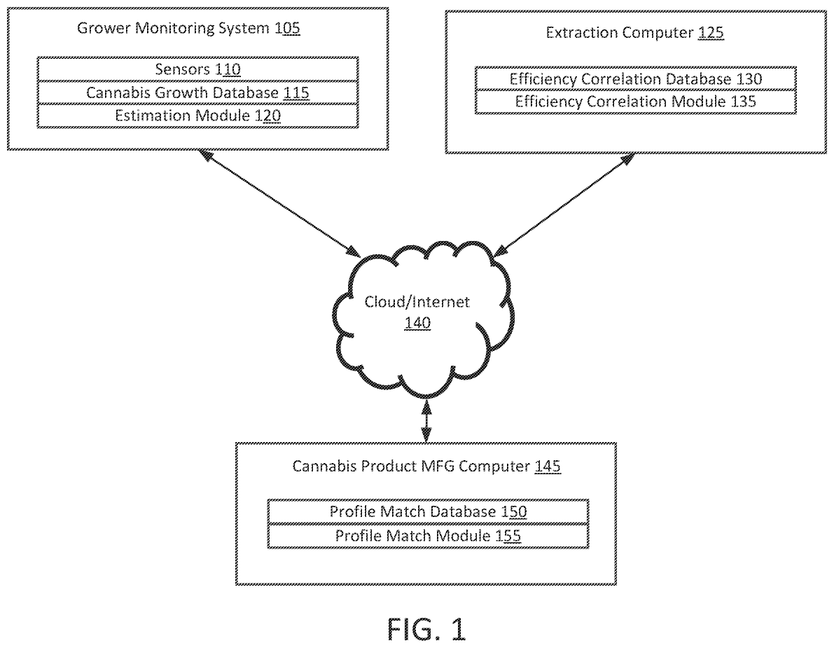

FIG. 1 illustrates computers of a grower, an extractor, and a cannabis product manufacturer that may communicate with each other when grow data is used to identify preferred extraction optimization metrics. The computers of FIG. 1 may also be used when an cannabis product manufacturer considers purchasing concentrates that have been or will be produced by an extractor. FIG. 1 includes grower monitoring system 105, extraction computer 125, and cannabis product manufacturer computer 145 that may communicate with each other over the cloud or Internet 140. Grower monitoring system 105 includes sensors 110, cannabis growth database 115, and estimation module 120. Grower monitoring system 105 may receive sensor data from sensors 110 that reside at a grow site. A processor executing instructions consistent with estimation module 120 may identify an amount of plant matter biomass (e.g. number of bushels or mass of cannabis plant biomass measurable in kilograms) currently growing in a field or an indoor grow facility (e.g. a greenhouse) and may estimate a total amount of plant matter biomass that will be available at harvest time. These estimates may also include a total number of specific cannabinoids that should be included in the grower's plant matter at harvest time (e.g. cannabinoid content, cannabinoid mass per unit volume of plant matter, or cannabinoid mass per estimated plant biomass).

Grower monitoring system 105 is a system that monitors the growth of cannabis through monitoring sensors 110. Grower monitoring system 105 may store sensor data, plant growth estimates, or other relevant growth conditions in the cannabis growth database 115. Grower monitoring system 105 may also send plant data or estimated yield data to extractor computer 125 or to cannabis product MFG computer 145. Estimates of plant growth may be made by performing calculations that use growth factors. Such a growth monitoring system may include different types of sensors. In one instance, optical sensors or cameras may be used to identify plant height, an overall plant area, or volume of plant material. Alternatively or additionally, optical sensors collect light spectra of plant matter such that a hyper spectral analysis or a multi spectral analysis may identify be used to identify plant conditions. Other sensors may include moisture or sunlight sensors. In order to provide estimated yields at harvest time, data may be collected continuously such that changes to estimates may be identified as the cannabis plants grow through their lifecycles. These estimates may identify a total number of kilograms (kg) of plant matter that will likely be harvested from a grow site or may identify in a mass of a type of concentrate that will likely be produced from a given set of plant matter. Extraction computer may provide information to computers of growers or cannabis product manufacturer s as part of a feedback network that allows growers to improve their growing techniques for optimal extraction.

Monitoring sensors 110 are sensors that monitor cannabis growth conditions (e.g. plant size, water frequency, plant density, soil acidity, temperature, light condition, light cycles, humidity, nutrients etc.). Sensors 110 for example may be optical sensors that can identify plant heights, plant area, or volumes of plant matter. In certain instances optical sensors may be capable of performing hyper spectral analysis or multi-spectral analysis. This sensor data may be used to identify when plants are ready to be harvested. Other sensors may be used to identify humidity, moisture, or sunlight. Various different sets of sensor data may be used to estimate a total volume of plant matter growing in a field. Cannabis growth database 115 may be a database that stores information that identifies a type of cannabis biomass (e.g. Cannabis sativa, Cannabis indica, hemp, etc) or a cannabis cultivar (e.g. cultivar name, for example Sour Diesel). Cannabis growth database 115 may also identify corresponding plant growth conditions (e.g. plant size, water frequency, plant density, soil acidity, temperature, light condition, light cycle, humidity, nutrients etc.). This growth data and estimates may be stored in cannabis growth database 115 and this data may be shared with extraction computer 125 such that the extraction computer can identify preferred extraction metrics or processes for extracting cannabinoids from the grower's plant matter.

Extraction computer 125 of FIG. 1 includes an efficiency correlation database 130 and an efficiency correlation module 135. The efficiency correlation module 135 of FIG. 1 may be a set of program code instructions that are executed by a processor. In certain instances, extraction computer 125 may help improve the efficiency of the extraction process by collecting data over time. Extraction computer 125 may optionally feedback estimates to growing monitoring system 105 such that a grower may be made aware of an estimated efficiency of an extraction process. This information fed back to the grower may identify an estimated mass of extract or an expected extract quality. The grower may also review data received from the extraction computer 125 when identifying how best to grow certain types of cannabis plants to optimize extract yield. As such, the grower may be able provide growth conditions that are correlated to efficient extraction and extraction yield. Execution of the instructions of the efficiency correlation module 135 may cause the processor of extraction computer to match a type of cannabis plant matter biomass with extraction data from previous extractions of similar cannabis plant matter biomass. Data from previous extractions may be stored in efficiency correlation database 135. As such efficiency correlation database 135 may store sets of data that were identified using a series of experiments that can be used to correlate a specific type of plant biomass with a preferred extraction process. After the processor of extraction computer 125 has identified an estimated number of cannabinoids and types of specific cannabinoids included in cannabis plant matter and identified a preferred extraction process, the processor at extraction computer 125 may identify extraction efficiencies and a total number of cannabinoids that should be yielded from a plant matter extraction. These estimates may also include an estimated total volume of a concentrate (e.g. liters of a distillate or an isolate) output from an extraction process.

Efficiency correlation database 130 may store historical data that cross references data from growers with data that describes an efficiency of an extraction process. Such efficiency data may include a mass of an extract produced by an extraction process, may identify an extract quality, or may identify an extract purity level. Database 130 may also store information used to identify types of cannabis plants and may also store information that identifies specific growers. In certain instances, a cannabis input type or particular cultivar may be cross-referenced with relevant growth conditions that are correlated to efficient parameters for extracting cannabinoids from specific types of cannabis biomass. In certain instances, data stored at extraction computer 125 of FIG. 1 may include data from different extractors that use different extraction methods and different process parameters.

Products made from extracts by a cannabis product manufacturer may be known as cannabis derivative products or cannabis products that may be in the form of a consumable item or may be in the form of a balm, rub that may be applied to the skin as a topical agent, or may be in the form of a cannabinoids that have been prepared for vaporization. As such, cannabis edibles may be in the form of a food product such as a chocolate, a cookie, or as a beverage or as an oil intended to be ingested, such as in a capsule. Other cannabis products may be in a form that is readily vaporized in a vaporizer or that is in a form that is readily absorbed through the skin. Estimates regarding total concentrate yield may then be shared with cannabis product MFG computer 145 via the cloud or internet 140. Cloud or Internet 140 is the global system of interconnected computer networks that use the Internet protocol suite (TCP/IP) to link devices worldwide. It is a network of networks that consists of private, public, academic, business, and government networks of local to global scope, linked by a broad array of electronic, wireless, and optical networking technologies. The Internet carries a vast range of information resources and services, such as the inter-linked hypertext documents and applications of the World Wide Web (WWW), electronic mail, telephony, and file sharing. Estimates of cannabinoid content included in a concentrate may be compared to a profile stored at cannabis product MFG computer 145 when program code of profile match module 155 is executed by a processor at cannabis product MFG computer 145. When the processor identifies that a concentrate estimate is consistent with a product profile, the cannabis product MFG computer 145 may send a message to buyers at the cannabis product manufacturer such that concentrates may be preordered. In certain instances, buyers at a cannabis product manufacturer may sign future contracts with growers well before a concentrate has been produced. These buyers may also contract to purchase a distillate or an isolate that contains certain cannabinoids. After an cannabis product manufacturer receives an extract, they may use that extract to make cannabis products that may again be in the form of an edible, a topical cream, or a vaping extract.

Cannabis product MFG computer 145 may be a platform that receives information from the grower monitoring system 105 on the types and cultivars of cannabis grown, plant growth conditions, and estimated yields. Program code of profile match module 155 may match the profiles of cannabis concentrates with the products that an cannabis product manufacturer wishes to make. Operation of the program code of the profile match module 155 may send messages or prompts to buyers of cannabis product manufacturer s to inform those buyers of concentrates that will be available for purchase. In certain instances, these messages may identify changes in the amount or type of cannabis concentrates that may be available for purchase as plant conditions change.

Edible manufacture profiles may identify a type and profile of a cannabis concentrate suitable to manufacture particular products. The type of cannabis concentrate may include for example a liquid extract containing a specific cannabinoid, concentration of cannabinoids or ratio of cannabinoids suitable for particular products. Operation of the profile match software module 155 may match cannabinoids that should be included in an edible product with concentrates that are or should be available for purchase. In certain instances, data stored in the profile match database 150 may be compared with current growth conditions and estimated yields of the type(s) and/or cultivar(s) of cannabis plants. This may allow an cannabis product manufacturer to identify and purchase cannabis concentrates that are consistent with a desired profile. In certain instances, edible manufacture computer may identify that changes to growing conditions may require a change to a manufacturing process. For example, in an instance when Mr. Brown's Cookies requires a cannabis concentrate to have a THC to CBD ratio of 75% to 25% to manufacture a cookie, cannabis product MFG computer 145 may identify that plant biomass number 007 could be used as an additive to a cookie manufacturing process when plant biomass number 007 includes a THC to CBD ratio that matches the 75% to 25% requirement. In such an instance, a manufacturer that produces Mr. Brown's Cookie may be sent a notification when an estimated yield of cannabis concentrate from biomass 007 may drop rapidly due to unforeseen weather conditions. Such a notification may identify similar cannabis concentrates that match the 75% to 25% THC to CBD profile required to make Mr. Brown's Cookies. Based on such a notification, the manufacturer of Mr. Brown's Cookies may change a manufacturing schedule, contact new extractors, contract with other growers, or adjust their manufacturing procedures such that their product can be manufactured.

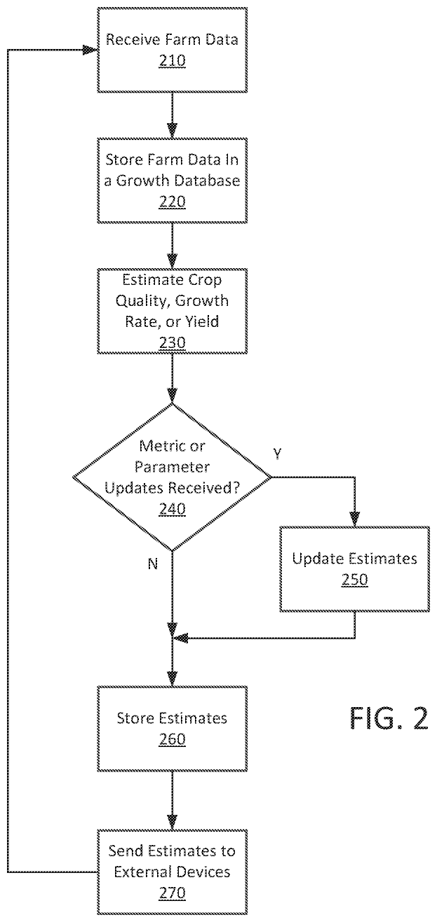

FIG. 2 illustrates a series of steps that may be performed by a computer that collects data at a cannabis grow site (e.g. a farm, a greenhouse, or an indoor growing facility). FIG. 2 begins with step 210 where grow data is received at a grower computer, such as grower monitoring system 105 of FIG. 1. This grow data may be received from a set of grow sensors that measure one or more of ambient temperatures, humidity in the air, soil moisture content, soil chemical levels, soil PH (e.g. an acid level or a base level), a volume of water provided to cannabis plants, or an amount of rainfall. The grow data may also identify a type of cannabis plant biomass or may identify plant height, average light hours per day, average humidity, or normalized yield. The sensor data collected overtime may be used to identify preferred conditions for growing cannabis plants. The grow data received in step 210 may also include data received from cannabis sensors or data that was the result of an analysis that used cannabis sensor data or a combination of cannabis sensor data and grow sensor data. Cannabis sensors may be sensors that measure data from which plant specific data can be derived. Examples of plant specific data include plant height, plant biomass, plant biomass density, plant matter volume, average trichome density, cannabinoid content, cannabinoid mass per unit volume of plant matter, or cannabinoid mass per estimated plant biomass.

The grow data received in step 210 may be stored in a grower database in step 220 of FIG. 2. Next, in step 230 of FIG. 2 an estimated crop quality, an estimated growth rate, or a yield may be estimated. Such estimates may be based on a stage of a life cycle of cannabis plants. Life cycle stages of cannabis plants may include seed, sprout, seedling, juvenile from seed, adolescent from seed, adult from seed, adult from seed and seed producing, clone, juvenile clone, adolescent clone, adult clone, and mother of clones. Alternatively, the lifecycle of cannabis plants may be classified as germination, seedling, vegetative from seed, flowering from seed, juvenile clone, vegetative clone, and flowering clone. Each one of these life cycles may be associated with specific metrics or parameters for identifying a quality, a growth rate, or a projected yield. The yield analysis may also consider effect of plant diseases, accidental plant damage, plant rot, root rot, mold, humidity, soil PH, or consider any factor discussed below in respect to table 1.

Data collected over time or expected data may be organized in a data structure like table 1 below that may be used to cross-reference and track the growth of specific plants as plants grow through their life cycle stages. The life cycle stages included in table 1 are germination, seedling, vegetative, and flowering. The metrics included in table 1 are life cycle stage time, temperature range, humidity, soil moisture, soil chemical content, soil PH, fertilizer applied, volume of water applied per unit time, rain volume per unit time, height/length, plant biomass, biomass density, plant matter volume, trichome density, cannabinoid content, and growth rate. Initially certain cells of table 1 may be populated with estimates that may include preferred baseline durations of plant life cycle, preferred temperature ranges, a preferred humidity, and other preferred baseline metrics. As time goes on, table 1 may also be populated with actual measured or interpolated data. Overtime, best case metrics may be identified. For example, best case metrics may include temperatures, volumes of water, soil moisture content, and soil chemical content that result in fastest plant growth. Plant growth may be measure by a length of time that a particular plant has stayed in a particular growth stage, a plant height/length, a total amount of actual or estimated plant biomass, a plant matter volume, a biomass density, a trichome density, a cannabinoid content, or a growth rate (mass, density, or height change per unit time). Each different stage of a plant's lifecycle may be associated with different preferred metrics over time and these identified preferred metrics may replace metrics that were originally used as baseline metrics. As such, plant growth can be characterized and then optimized to maximize cannabinoid yield over time.

TABLE 1
Cannabis Lifecycle vs. Farm/Plant Metrics

| Cannabis Life | | | | |
|---|---|---|---|---|
| Cycle Stage vs. | Germination | Seedling | Vegetative | Flowering |
| Grow & Plant | Baseline vs. | Baseline vs. | Baseline vs. | Baseline vs. |
| Metrics | Actual | Actual | Actual | Actual |
| Life Cycle Stage | 0-1 week vs. XX | 1-3 weeks vs. | 3-12 weeks vs. | 12-18 weeks vs. |
| Time | | | | |
| Temperature | 70-80 vs. | 70-80 vs. | 70-90 vs. | 65-90 vs. |
| Range Degrees F. | | | | |
| Humidity | 60% vs. | 50% | 45% | 45% |
| Soil Moisture | | | | |
| Soil Chemical | | | | |
| Content | | | | |
| Soil PH | | | | |
| Fertilizer App. | | | | |
| Volume of | | | | |
| Water/unit Time | | | | |
| Rain Volume/unit | | | | |
| time | | | | |
| Fog Density | | | | |
| Height/Length | | | | |
| Pant Biomass | | | | |
| Biomass Density | | | | |
| Plant Matter | | | | |
| Volume | | | | |
| Trichome Density | | | | |
| Cannabinoid | | | | |
| Content | | | | |
| Growth Rate | | | | |

After estimates are made in step 230, determination step 240 may identify whether any metrics or parameters should be updated. In certain instances, a grower may provide updated metrics or parameters to program code that generates the estimates. For example, a grower may indicate that he provided his plants with an additional volume of water or with additional fertilizer. Alternatively, program code may be developed that automatically changes a parameter used in an equation that forecasts future plant growth. For example, if an estimate of plant growth after a fertilizer application does not correspond to actual plant growth measurement data, a parameter in the plant growth forecasting equation could be changed such that the equation would generate growth rate estimates that corresponded better to the actual measurement data. Such a parameter change may be identified by re-running estimates using data recorded from an earlier time. For example, if a plant was forecast to grow in a week by six inches in height after a fertilizer application and the plant actually grew eight inches in height, a parameter in the growth rate forecasting equation could be increased to a value where the re-running of the last growth rate forecast would provide an eight inch height growth forecast instead of the six inch growth forecast. When determination step identifies that growth estimates should be updated, program flow may move from step 240 to step 250 of FIG. 2 where the estimates may be updated. Next, those updated estimates may be stored in a database in step 260 of FIG. 2. When determination step 240 of FIG. 2 identifies that estimates should not be updated, program flow may move from step 240 to step 260 where estimates made in step 230 may be stored in the database. After step 260, the estimates may be sent to computing devices that are external to a growing monitoring system or computer in step 270 of FIG. 2. For example, when the growing monitoring system 105 of FIG. 1 makes a plant quality, growth rate, or yield estimate, that estimate may be sent to cannabis product MFG computer 145 or extraction computer 125. The data sent in step 270 may be used to identify an estimated market price for a grower's plants at harvest time or to update a price that was estimated earlier.

Note that metrics associated with temperature, humidity, soil moisture, soil PH, rain volumes per unit time, fog density, biomass density, plant biomass, or trichome density may be measured or be estimated from sensor data. Some of these factors, such as temperature, humidity in the air, soil moisture, soil PH, rain volume, and fog density may be measured directly using a particular type of sensor. Other of these factors, such as biomass density, plant biomass, or trichome density may be estimated by data collected by other sensors. An amount of biomass may be estimated from image data. Cannabis plants in a vegetative state typically have clusters of leafs that are attached to a single stem at a base portion of what may be referred to as a “leaf cluster.” Such leaf clusters typically include an odd number of individual leafs that draw nutrients through the single stem that attaches them to a branch or a main stem of a cannabis plant. A plant in a vegetative state that has leaf clusters separated by a centimeter could be assigned a “low leaf plant density metric,” vegetative plants with clusters of leafs that are separated by less than a centimeter could be assigned a “medium leaf plant density metric,” and vegetative plants with overlapping leafs on a plant that light does not readily shine through may be assigned a “high leaf plant density metric.” Similarly, flower material could be assigned different metrics based on a trichome density. Such a trichome density could correspond to a count of trichomes in an image combined with an estimated volume of a flower or number of flowers. Trichome densities could also correspond to a total length along a stem that is covered with plant flowers (buds).

Table 2 illustrates other growth data that may be stored in a grower database, such as the cannabis growth database 115 of FIG. 1. Table 2 includes a first column that identifies biomass type and a plant lot reference number, a second column that identifies a number of days included in current growth cycle, a third column that identifies an amount of water provided to a given plant lot number, and a fourth column that identifies an estimated plant density. Table 2 also includes different columns that identify soil PH, average ambient temperature in degrees Celsius (C), numbers of average hours of light provided to the plants, average ambient humidity levels, and normalized yield estimates.

TABLE 2
Cannabis Growth Data

| | | Plant | | | | | | Daily | | |
|---|---|---|---|---|---|---|---|---|---|---|
| Biomass | Growth | Avg. | Plant | Daily | | | Avg. | Avg. # | | |
| Type & | Cycle | Height | Area | Water | Plant | Soil | Tmp. | of light | Avg. | Norm |
| Number | Days | (FT) | Sq FT | Vol. | Density | PH | C. | hours | Humidity | Yield |
| Sativa | 2 | 0.01 | 21000 | 4000 | 20% | 5.8 | 26 | 23 | 40% | 10% |
| 101 | | | | | | | | | | |
| Hemp | 68 | 1.8 | 11000 | 4300 | 19% | 6.2 | 24 | 19 | 60% | 51% |
| 102 | | | | | | | | | | |
| Indica | 80 | 2.6 | 15500 | 6300 | 60% | 5.5 | 21 | 12 | 65% | 80% |
| 103 | | | | | | | | | | |
| Sativa | 95 | 2.5 | 17500 | 6400 | 65% | 6.1 | 21 | 10 | 40% | 92% |
| 104 | | | | | | | | | | |
| Hemp | 105 | 3.0 | 21500 | 7200 | 70% | 5.9 | 23 | 10 | 55% | 95% |
| 105 | | | | | | | | | | |

Note that the types of biomass included in table 2 include Cannabis sativa, hemp, and Cannabis indica. Note that the different lots of cannabis sativa have been provided lot numbers of 101 and 104, that the different lots of hemp has been provided lot numbers of 102 and 105, and that the lot of cannabis indicia has been provided lot number 103. Each of the plants in each of these different lots of plants have been growing a different number of days. The data of table 2 also indicates that as the plants age, they grow taller, and require more water. This data also indicates that as the plants age their density also tends to increase and that after the plants age, they may be provided less light. The 23 hours of light indicated in the first row of table 2 may be the result of a grower providing artificial light to the plants of lot 101. Furthermore, the shorter light cycles may be the result the removal of light or the tenting of plants. From the density data or other data, a processor at a computer may calculate an estimated normalized yield. As the plant matures, this yield estimate may increase. In certain instances, the yield estimate may increase with plant density.

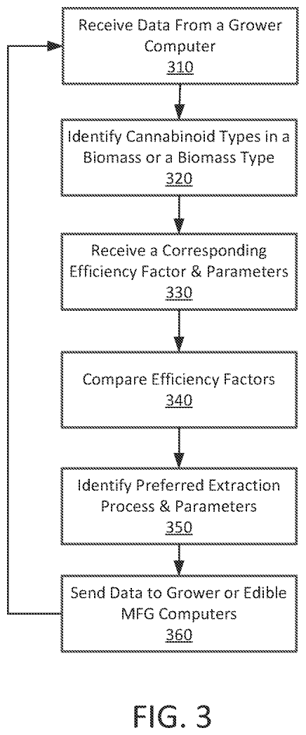

FIG. 3 illustrates exemplary steps that may be performed when preferred extraction process parameters are identified for extracting cannabinoids from specific types of cannabis plants. The steps of FIG. 3 may be performed by an extraction computer, such as the extraction computer 125 of FIG. 1. FIG. 3 begins with step 310 where data may be received from a grower monitoring system computer. The data received in step 310 may include cannabis growth estimates collected by the steps of FIG. 2. As such, the data received in step 310 may include an estimated crop quality, growth rate, or yield. The data received in step 310 may also include information that identifies or that can be used to identify a type of cannabis plant biomass. Next, in step 320, a processor at the extraction computer may identify the type of cannabis plant biomass from the received data or by parsing test data. A particular type of biomass or cannabinoids included in that biomass may be identified based on inputs that were originally received at a grower computer, by accessing test results stored at the grower computer, or by accessing test data stored at a computer of a test lab. These data may also include cannabinoid content, cannabinoid profile and other chemical and physical properties of the plant biomass. Testers that may have originally acquired this test data may be any tester using any analytical technique known in the art including, yet not limited to an optical tester, a high performance liquid chromatograph (HPLC), an ultra-high performance liquid chromatograph (UHPLC), a gas chromatograph (GC), a spectral tester, or other type of chromatograph or analytical technique. When the type of biomass and biomass quality is identified by a test, test results may be received by a computer directly from a tester or from a database of a test lab that tested and analyzed the sample of the plant biomass.

Cannabis sensors can include cameras, optical sensors, or density sensors. In some instances, the soil PH, moisture, or chemical sensors classified above as grow sensors may be classified as cannabis sensors. Data from one or more cameras may be used to identify the height of different plants in a field and may also be used to identify whether the plants appear to be filling in with leaf or flower plant matter as expected. An analysis of this camera data may also be used to identify a general density of plant matter. For example, camera data could be used to classify plants into categories of high, medium, and low density. The assignment of a general density category to a plant may be a function of plant age, plant height, plant type, or other metrics. General density may also be identified based on identifying how much flower matter or space on the plant that includes stem and no plant matter. Optical sensors may collect spectral or hyperspectral data from plants growing in the field. From this sensor data, grower computer 120 may identify health metrics (e.g. high, medium, low) to assign to the plants or to identify a cannabinoid content included in the plants. The health metrics may be identified by identifying colors included in light reflected by the plants or colors of light shined through a plant biomass. The assignment of a health metric to a plant may also be a function of plant age, plant height, plant type, or other metric combined with spectral or hyperspectral data. Methods consistent with the present disclosure may be used to identify sets of environmental characteristics that cause certain types of cannabis plant to grow more rapidly. Additionally or alternatively, optical sensors that may be used with the present invention include any type of camera or device that acquires images, senses reflected light, or senses an amount of light that passes through a sample of plant matter. Collected image data may be used to identify colors of trichomes when identifying or estimation a number or types of cannabinoids that may be included in plant material. Image data may be used to identify a height of different plants at the farm.

After step 320, step 330 may identify an efficiency factor or parameters associated with extracting specific cannabinoids or with extracting cannabinoids from the type and properties of identified cannabis plant biomass. This efficiency factor may be a coefficient in an equation used to estimate extraction efficiency (e.g. the percentage recovery of available cannabinoids and other compounds from the plant biomass through the extraction process). Efficiency factors may be retrieved from a database such as the efficiency correlation database 130 of FIG. 1. Extraction efficiencies may be estimated by execution of program code of efficiency correlation module 135 by a processor at extraction computer 125 of FIG. 1. Parameters retrieved in step 330 may be settings that affect how an extraction system operates. Such parameters may identify an extraction method and extraction conditions employed. Such parameters may for example identify a solvent type, microwave energy level, extraction temperatures, a ratio of solvent volume to plant mass, extraction time (e.g. length of time that cannabis biomass resides (residence time) in a continuous flow extraction chamber. Note that table 3 includes examples of these parameters. The data of table 3 may also be used to cross-reference different cannabis types with different biomass lot numbers, with different extraction parameters, and with different extract potencies and efficiency rates. Table 3 includes three different lots of high THC cannabis, two different lots of high CBD cannabis and two different lots of low THC hemp. A higher efficiency rate in table 3 for a given cannabis plant biomass type may be used to identify a preferred set of parameters for extracting cannabinoids included in a given type of cannabis plant biomass. The high THC cannabis of lot number T104 was assigned a 98% efficiency rate used parameters of Low microwave energy density and 12 minutes of residence time when ethanol was used as an extraction solvent at a ratio of 12 liters per kg. High THC cannabis lots T001 and T002 resulted in lower efficiency rates. As such, high THC cannabis lot T104 had the best extraction efficiency rate. Similarly, high CBD Cannabis lot C220 had the highest extraction efficiency rate and low THC hemp lot H001 had the highest efficiency rate. Step 340 may compare extraction efficiency rates as reviewed above and preferred extraction process parameters may be selected for a new lot of cannabis plant biomass in step 350 based on the comparison of the efficiency factors. As such, when the new lot of cannabis plant biomass is high THC cannabis, extraction parameters used to extract cannabis lot T104 may be used to extract the new lot of cannabis plant biomass because historically, those parameters resulted in best extraction efficiencies for high THC cannabis. Alternatively, when the new lot of cannabis plant material is low THC hemp, extraction parameters consistent with cannabis plant material lot H001 may be selected because historically, those parameters resulted in best extraction efficiencies for low THC hemp.

Next, in step 340 each of these different efficiencies may be compared and preferred extraction process parameters may be identified in step 350 by reviewing historical data. In certain instances, data stored at extraction computer 125 of FIG. 1 may include data from different extractors that use different extraction methods that may also require different process parameters. In certain instances, different extractors may use processes that are similar, yet use different solvents. Finally, in step 360 of FIG. 3, yield projections, preferred extraction process parameters, and efficiency rates data may be sent to computers of a grower that is growing the cannabis or to a cannabis product manufacturer that is interested in purchasing cannabis concentrates. This data could help the grower to negotiate a contract with the cannabis product manufacturer. After step 360, program flow moves back to step 310 of FIG. 3.

TABLE 3
Cannabis Extraction Process Parameters vs. Process Efficiencies

| | Lot | | | | | |
|---|---|---|---|---|---|---|
| | Number/ | | | | | |
| Cannabis | Biomass | | Microwave | Solvent to | | |
| Biomass | Reference | | Power | Biomass | Residence | Efficiency |
| Type | Number | Solvent | Density | Ratio (l/kg) | Time (min) | Rate |
| High THC | T001 | Ethanol | Low | 10 | 5 | 93% |
| Cannabis | | | | | | |
| High THC | T002 | Pentane | Med | 10 | 5 | 97% |
| Cannabis | | | | | | |
| High THC | T104 | Ethanol | Low | 12 | 20 | 98% |
| Cannabis | | | | | | |
| High CBD | C220 | Ethanol | High | 12 | 10 | 94% |
| Cannabis | | | | | | |
| High CBD | C301 | Ethanol | Low | 12 | 10 | 87% |
| Cannabis | | | | | | |
| Low THC | H001 | Ethanol | Med | 12 | 20 | 83% |
| Hemp | | | | | | |
| Low THC | H202 | Ethanol | Low | 10 | 20 | 71% |
| Hemp | | | | | | |

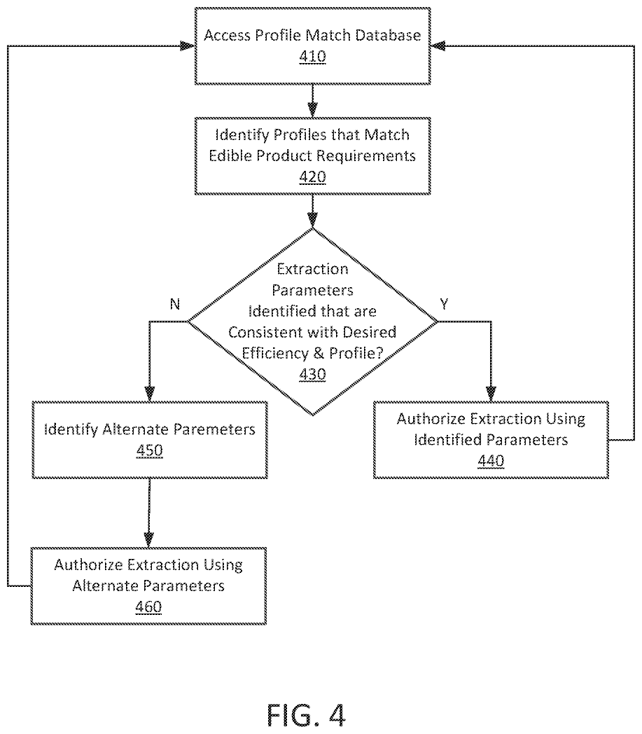

FIG. 4 illustrates a series of steps that may be performed by a computer when parameters for extracting cannabinoids from cannabis plant biomass are selected. FIG. 4 begins with step 410 where a profile match database is accessed by an extraction computer. Extraction computer 125 of FIG. 1 may access the profile match database 150 of cannabis product MFG computer 145 when collecting information consistent with edible products that an cannabis product manufacturer intends to produce. Step 410 may retrieve a set of profiles consistent with the manufacture of one or more edible products that an cannabis product manufacturer produces. These profiles may include a type of cannabis concentrate (e.g. liquid oil containing a specific concentration of THC), or a required THC to CBD ratio and a volume of an extract within which a mass of THC and a mass of CBD should fill. For example, an edible product profile may identify that a particular product should include a THC to CBD ratio of 80% THC to 20% CBD, that the product should include 8 milligrams (mg) of THC and 2 mg of CBD, and that the volume of extract that contains the 8 mg of THC and the 2 mg of CBD should fit in a volume of 0.1 milliliters (0.1 cubic centimeters). This product profile data could be used to identify whether the extraction and concentration processes should produce a distillate or an isolate. In instances where the volume allocated to the extract in the profile is too small for a distillate to be used, then a selected extraction and concentration process may require that an isolate be produced. After the product profile data has been received, a processor at the extractor computer may identify a product profile that matches a product profile requirement. Next, determination step 430 may identify whether extraction parameters stored in a database at the extractor computer are consistent with a desired extraction efficiency and with the product profile, when yes program flow may move from determination step 430 to step 440 of FIG. 4. Step 440 may then authorize an extraction to be performed on cannabis plant matter using the identified parameters. After step 440, program flow may move back to step 410 of FIG. 4. When determination step 430 identifies that extraction parameters stored at the extraction computer are not consistent with desired efficiencies and the product profile, program flow may move to step 450 where alternate extraction parameters may be identified. The identification of these alternate extraction parameters may be performed by various different processes that may include estimating changes to extraction parameters using interpolation or an averaging process. Alternate extraction parameters may also be estimated by selecting parameters that are consistent with a minimum or maximum range or by performing a calculation that extrapolates a result.

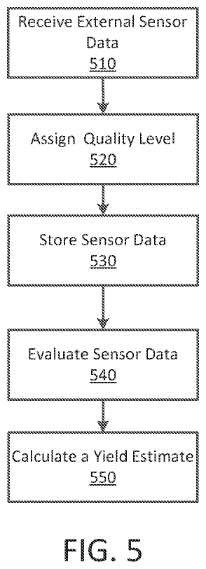

FIG. 5 illustrates an exemplary set of steps that may be performed by a control system that calculates yield estimates from grow data. FIG. 5 begins with a step 510 that receives sensor data that may have been sensed by sensors at a farm. These sensors may include sensors that sense cannabinoid content or that sense a density of trichomes of cannabis plant matter. Next, in step 520 of FIG. 5, a computer may assign a quality level to the plant matter. Trichomes in cannabis plant matter are hairy structures that create cannabinoids and color changes may indicate plant maturity and changes in cannabinoids included in specific trichomes.

This quality level identified in step 520 may be assigned based on a number of milligrams of a cannabinoid are included in an average gram of cannabis plant matter growing at a farm. As such, a quality level may be a percentage of cannabinoids included in a mass of the cannabis plant matter on average. Optical sensors may identify a density of trichomes in a unit area of plant matter. In one instance, images or video of plant matter may be acquired as cannabis plant matter grows in a field. A processor executing instructions out of a memory may identify a number of trichomes per unit area (e.g square millimeters or centimeters) on leaf surfaces or per unit volume (e.g. cubic millimeter or centimeters) in flower material. Additional tests may be performed on leaf or flower material to identify a mass and an area of representative leaf samples or the mass and volume of representative flower samples. These additional tests may identify a mass of cannabinoids included in the leaf samples and in the flower samples. Testers that perform these tests may use any analytical technique known in the art including, yet not limited to an optical tester, a high performance liquid chromatograph (HPLC), an ultra-high performance liquid chromatograph (UHPLC), a gas chromatograph (GC), a spectral tester, or other type of chromatograph or analytical method. An analysis of images of the various samples may allow for the processor to identify a number of trichomes included in an area of leaf material and a number of trichomes included in a volume of flower material. The processor may review image data when identifying or estimating a total area of leaf matter and a total volume of flower matter that is included in a set of plants. If the leaf matter were identified to include 1 mg of THC per square centimeter and the flower material were identified to have 30 mg of THC per cubic centimeter the processor could evaluate the received image data to estimate a total number of square area of leaf material and a total volume of flower material included in the plants. Assume that a set of plants has a volume of 5,000 cubic centimeters that includes 4000 cubic centimeters (cm) of flower material and 1000 square centimeters of leaf material, then an estimate could be made of a total mass of THC included in the tote could be calculated: 4000 (cubic cm of bud material)*30 (mg/cubic cm)+1000 (square cm of leaf material)*1 (mg/square cm)=120,000 mg THC+1000 mg THC=121,000 mg of THC (or 121 g or 0.121 kg of THC). A total weight of combined material could be identified by weighing cut plant matter. Furthermore, the processor could calculate an estimated weight of flower material and leaf material included in the plants. The processor could then execute instructions to identify whether the estimated weight was within a threshold percentage of the total weight. When the estimated weight was within a threshold distance of the actual weight (e.g. 3%), the processor could calculate a total mass of THC included in the plants. The estimate made by plant material weight calculations may be compared to the estimate made by area or volumetric calculations. Data relating to both of these estimates may be stored in a database and values of these estimates may be compared to each other to see if they are within a threshold distance of each other (e.g. within 2%). Such processes could help identify, manage, control, or update the ways in which estimates were made when an estimate derived from trichrome area/volume equations was not consistent with (e.g. a greater that 2% difference) with an estimate derived using mass equations.

In certain instances, the processor may review the image data to identify color and density of trichomes when identifying whether their color and density appeared consistent with the material samples discussed previously. The processor could receive an image of a flower when estimating a total number of cannabinoids included in that flower. This estimate could be based on a number of trichomes observed, test sample data, an estimated volume of the flower, color or contrast of the trichomes, and or an estimated mass of the flower. If the colors of trichomes in a flower are identified as not being fully developed, a total estimated mass of cannabinoids included in the flower may be de-rated by a derating factor. For example, when the flower is cloudy, white colored, or opaque, the flower may be considered high quality and fully developed. When the trichomes in the flower are clear (translucent), the flower may be considered immature and have a low quality. When the flower contains an even distribution of clear and cloudy trichomes, it may be associated with a medium quality level. Amber, orange, or brown colored trichomes may be associated with, yet another quality or classification that may indicate higher cannabinol (CBN) levels or a greater likelihood that consumption of these amber, orange, or brown colored trichomes will induce a “couch lock” effect. The “couch lock” effect is an effect reported by people that consume cannabis that makes them sleepy. This effect may be associated with cannabis that includes higher levels of CBN as CBN is believed act as a sedative to those that consume it. The colors, clarity, or opaqueness of cannabis plant trichomes described above are representative and are not intended to limit the scope of the present disclosure. Quality assignments based on colors, clarity, or opaqueness of cannabis plant trichomes may be updated overtime as data is collected. For example, it may be found that when about 60% of the trichomes are opaque, 30% of the trichomes are amber, and when less than 10% of the trichomes are clear result in a better quality extract for a given extraction process or set of extraction parameters. Furthermore, any color spectra of plant trichomes may be identified as providing a better quality extract or an improved extraction efficiency (increased yield) for a given extraction process over time. As such, methods consistent with the present disclosure allow an extractor to learn how to identify preferred materials and how to set process parameters to perform more efficient extractions. Densities of trichomes may also be identified by a sonic or ultrasonic sensor that senses density by identifying a measure of sonic or ultrasonic energy that has been absorbed by or reflected by samples of plant matter.

After the quality level is assigned in step 520, the received sensor data may be stored in a database in step 530 and that sensor data could be evaluated in step 540 of FIG. 5. The evaluation performed in step 540 may review the sensor or image data and compare that data with data from previous lots. Next, in step 550 of FIG. 5 a yield estimate may be calculated. This yield estimate may estimate a total number of bushels or Kg of plant matter growing in a field, may estimate a total mass of cannabinoids in the field, and may identify masses of cannabinoids that could be extracted from the plant matter. The steps performed in FIG. 5, could be performed by the extraction computer 125 of FIG. 1 after receiving growth data from grower monitoring system 105 of FIG. 1.

After a yield estimate has been made that includes a profile of cannabinoid content that should be include in an extract, this profile estimate may be compared to cannabis concentrate profile requirements for manufacturing edible products made by a manufacturer. Table 4 identifies that cannabis plant biomass 002 is suitable for making a concentrate that can be included in Hope cranberry juice that will include 50 mg of THC per 100 ml serving. Table 4 also identifies that cannabis plant biomass 031 is suitable for making a concentrate that can be included in Dr. Brown's cookies, where 1 cookie serving should contain liquid THC/CBD with a mass of THC of 25 mg and a mass of CBD of 10 mg.

TABLE 4
Edible Profile Data

| Biomass | | Cannabis Concentrate |
|---|---|---|
| Number | Edible Type | Profile |
| 002 | Hope Cranberry Juice 100 ml | Liquid THC, 50 mg |
| 031 | Dr. Brown Cookie, 1 serving | Liquid THC/CBD, 25 |
| | | mg/10 mg |

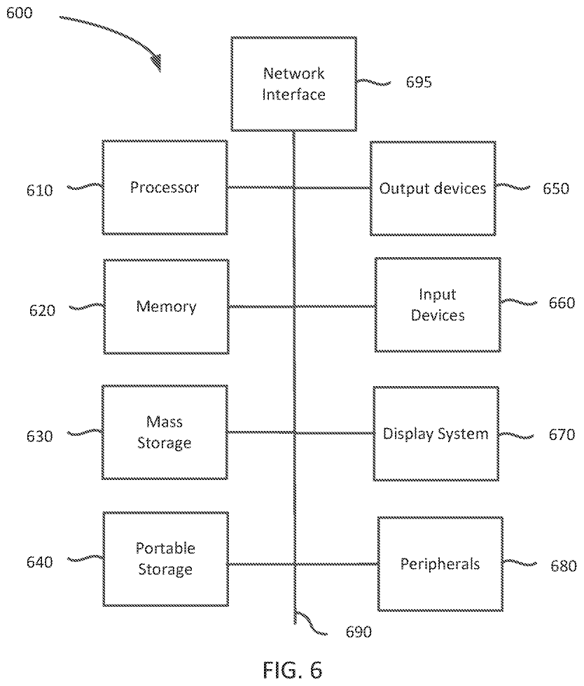

FIG. 6 illustrates a computing system that may be used to implement an embodiment of the present invention. The computing system 600 of FIG. 6 includes one or more processors 610 and main memory 620. Main memory 620 stores, in part, instructions and data for execution by processor 610. Main memory 620 can store the executable code when in operation. The system 600 of FIG. 6 further includes a mass storage device 630, portable storage medium drive(s) 640, output devices 650, user input devices 660, a graphics display 670, peripheral devices 680, and network interface 695.

The components shown in FIG. 6 are depicted as being connected via a single bus 690. However, the components may be connected through one or more data transport means. For example, processor unit 610 and main memory 620 may be connected via a local microprocessor bus, and the mass storage device 630, peripheral device(s) 680, portable storage device 640, and display system 670 may be connected via one or more input/output (I/O) buses.

Mass storage device 630, which may be implemented with a magnetic disk drive or an optical disk drive, is a non-volatile storage device for storing data and instructions for use by processor unit 610. Mass storage device 630 can store the system software for implementing embodiments of the present invention for purposes of loading that software into main memory 620.

Portable storage device 640 operates in conjunction with a portable non-volatile storage medium, such as a FLASH memory, compact disk or Digital video disc, to input and output data and code to and from the computer system 600 of FIG. 6. The system software for implementing embodiments of the present invention may be stored on such a portable medium and input to the computer system 600 via the portable storage device 640.

Input devices 660 provide a portion of a user interface. Input devices 660 may include an alpha-numeric keypad, such as a keyboard, for inputting alpha-numeric and other information, or a pointing device, such as a mouse, a trackball, stylus, or cursor direction keys. Additionally, the system 600 as shown in FIG. 6 includes output devices 650. Examples of suitable output devices include speakers, printers, network interfaces, and monitors.

Display system 670 may include a liquid crystal display (LCD), a plasma display, an organic light-emitting diode (OLED) display, an electronic ink display, a projector-based display, a holographic display, or another suitable display device. Display system 670 receives textual and graphical information, and processes the information for output to the display device. The display system 670 may include multiple-touch touchscreen input capabilities, such as capacitive touch detection, resistive touch detection, surface acoustic wave touch detection, or infrared touch detection. Such touchscreen input capabilities may or may not allow for variable pressure or force detection.

Peripherals 680 may include any type of computer support device to add additional functionality to the computer system. For example, peripheral device(s) 680 may include a modem or a router.

Network interface 695 may include any form of computer interface of a computer, whether that be a wired network or a wireless interface. As such, network interface 695 may be an Ethernet network interface, a BlueTooth™ wireless interface, an 802.11 interface, or a cellular phone interface.

The components contained in the computer system 600 of FIG. 6 are those typically found in computer systems that may be suitable for use with embodiments of the present invention and are intended to represent a broad category of such computer components that are well known in the art. Thus, the computer system 600 of FIG. 6 can be a personal computer, a hand held computing device, a telephone (“smart” or otherwise), a mobile computing device, a workstation, a server (on a server rack or otherwise), a minicomputer, a mainframe computer, a tablet computing device, a wearable device (such as a watch, a ring, a pair of glasses, or another type of jewelry/clothing/accessory), a video game console (portable or otherwise), an e-book reader, a media player device (portable or otherwise), a vehicle-based computer, some combination thereof, or any other computing device. The computer can also include different bus configurations, networked platforms, multi-processor platforms, etc. The computer system 600 may in some cases be a virtual computer system executed by another computer system. Various operating systems can be used including Unix, Linux, Windows, Macintosh OS, Palm OS, Android, iOS, and other suitable operating systems.

The present invention may be implemented in an application that may be operable using a variety of devices. Non-transitory computer-readable storage media refer to any medium or media that participate in providing instructions to a central processing unit (CPU) for execution. Such media can take many forms, including, but not limited to, non-volatile and volatile media such as optical or magnetic disks and dynamic memory, respectively. Common forms of non-transitory computer-readable media include, for example, a floppy disk, a flexible disk, a hard disk, magnetic tape, any other magnetic medium, a CD-ROM disk, digital video disk (DVD), any other optical medium, RAM, PROM, EPROM, a FLASH EPROM, and any other memory chip or cartridge.

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

While various flow diagrams provided and described above may show a particular order of operations performed by certain embodiments of the invention, it should be understood that such order is exemplary (e.g., alternative embodiments can perform the operations in a different order, combine certain operations, overlap certain operations, etc.).

What is claimed is:

1. A method for estimating extraction yield, the method comprising:
receiving growth data regarding a set of cannabis plants sent over a communication network from one or more grower devices associated with the set of cannabis plants, the growth data sensed by one or more sensors communicatively coupled to at least one of the grower devices;
identifying a type of the cannabis plants based on the received growth data detected by the sensors;
determining an amount of plant matter in the set of cannabis plants based on the identified type, wherein the plant matter is associated with one or more cannabinoids; and
generating an estimate regarding a mass of the cannabinoids that are extractable from the determined amount of plant matter.

2. The method of claim 1, further comprising sending the estimate regarding the mass of the extractable cannabinoids over the communication network to a designated recipient device.

3. The method of claim 1, further comprising receiving a plant profile for the identified type that identifies relative concentrations of the cannabinoids within the identified type, the plant profile received from a database that stores a plurality of plant profiles.

4. The method of claim 3, further comprising:
retrieving historical extraction data associated with the identified type, the historical extraction data retrieved from the database in memory;
identifying a set of extraction parameters from the retrieved historical extraction data that best corresponds to the plant profile; and
sending the identified set of extraction parameters to an extraction apparatus for use in extracting the cannabinoids from the plant matter of the set of cannabis plant.

5. The method of claim 4, further comprising:
extracting the cannabinoids from the plant matter of the set of cannabis plant in accordance with the identified set of extraction parameters;
testing a resulting extract of the cannabinoids; and
storing the test results in association with the growth data in the database.

6. The method of claim 5, further comprising confirming that test results indicate the resulting extract is consistent with the plant profile.

7. The method of claim 4, further comprising receiving a preference regarding extraction, wherein identifying the set of extraction parameters is further based on the received extraction preference.

8. The method of claim 1, wherein the cannabinoids include at least one of tetrahydrocannabinol (THC) and cannabidiol (CBD).

9. The method of claim 1, further comprising authorizing extraction of the cannabinoids from the set of cannabis plants based on the relative concentrations meeting a predefined threshold concentration of at least one of the cannabinoids.

10. A system for estimating extraction yield, the system comprising:
one or more sensors that sense growth data regarding a set of cannabis plants;
a growth database associated with one or more grower devices, the growth database storing the growth data regarding the set of cannabis plants; and
an extraction computer communicatively coupled to the one or more sensors that:
receives the growth data detected by the sensors over a communication network;
identifies a type of the cannabis plants based on the received growth data;
determines an amount of plant matter in the set of cannabis plants based on the identified type, wherein the plant matter is associated with one or more cannabinoids; and
generates an estimate regarding a mass of the cannabinoids that are extractable from the determined amount of plant matter.

11. The system of claim 10, wherein the extraction computer further sends the estimate regarding the mass of the extractable cannabinoids over the communication network to a designated recipient device.

12. The system of claim 10, further comprising a database that stores a plurality of plant profiles,
wherein the extraction computer further receives one of the plant profile for the identified type that identifies relative concentrations of the cannabinoids within the identified type.

13. The system of claim 12, wherein the extraction computer further retrieves historical extraction data associated with the identified type; and further comprising an extraction apparatus, wherein the extraction computer further identifies a set of extraction parameters from the retrieved historical extraction data that best corresponds to the plant profile and sends the identified set of extraction parameters to the extraction apparatus for use in extracting the cannabinoids from the plant matter of the set of cannabis plant.

14. The system of claim 13, wherein the extraction apparatus that extracts the cannabinoids from the plant matter of the set of cannabis plant in accordance with the identified set of extraction parameters; and further comprising a testing chamber that tests a resulting extract of the cannabinoids, wherein the database further stores the test results in association with the growth data.

15. The system of claim 14, wherein the extraction computer further confirms that test results indicate the resulting extract is consistent with the plant profile.

16. The system of claim 13, wherein the extraction computer further receives a preference regarding extraction, wherein the extraction computer identifies the set of extraction parameters further based on the received extraction preference.

17. The system of claim 10, wherein the cannabinoids include at least one of tetrahydrocannabinol (THC) and cannabidiol (CBD).

18. The system of claim 10, wherein the extraction computer further authorizes extraction of the cannabinoids from the set of cannabis plants based on the relative concentrations meeting a predefined threshold concentration of at least one of the cannabinoids.

19. A non-transitory, computer-readable storage medium having embodied thereon a program executable by a processor to implement a method for estimating extraction yield, the method comprising:
receiving growth data regarding a set of cannabis plants sent over a communication network from one or more grower devices associated with the set of cannabis plants, the growth data sensed by one or more sensors communicatively coupled to at least one of the grower devices;
identifying a type of the cannabis plants based on the received growth data detected by the sensors;
determining an amount of plant matter in the set of cannabis plants based on the identified type, wherein the plant matter is associated with one or more cannabinoids; and
generating an estimate regarding a mass of the cannabinoids that are extractable from the determined amount of plant matter.
